(19) United States
(12) Patent Application Publication
(10) Pub. No.: US 2026/0182882 A1
(43) Pub. Date: Jul. 2, 2026

(54) METHODS, SYSTEMS, AND APPARATUS FOR EVALUATING TEST DATA OF PELVIC FLOOR MUSCLE

(71) Applicant: PEKING UNION MEDICAL COLLEGE HOSPITAL, CHINESE ACADEMY OF MEDICAL SCIENCES, Beijing (CN)

(72) Inventors: Lan ZHU, Beijing (CN); Xue ZHANG, Beijing (CN); Juan CHEN, Beijing (CN); Wenjia LOU, Beijing (CN); Xiaohui WANG, Beijing (CN); Qi DING, Beijing (CN); Weixi YAN, Beijing (CN)

(73) Assignee: PEKING UNION MEDICAL COLLEGE HOSPITAL, CHINESE ACADEMY OF MEDICAL SCIENCES, Beijing (CN)

(21) Appl. No.: 19/375,350

(22) Filed: Oct. 31, 2025

(30) Foreign Application Priority Data
Dec. 26, 2024 (CN) 202411933842.2

(51) Int. Cl. A61B 5/22; A61B 5/296

(52) U.S. Cl. CPC A61B 5/227; A61B 5/296

(57) ABSTRACT

Disclosed is a method, system, and apparatus for evaluating test data of pelvic floor muscle. The method includes obtaining the test data of the pelvic floor muscle within a test period. The method includes presetting a threshold value. The method includes obtaining a first baseline image for type I pelvic floor muscle fibers and a second baseline image for type II pelvic floor muscle fibers. The method includes constructing a test curve corresponding to type I pelvic floor muscle fibers and a test curve corresponding to type II pelvic floor muscle fibers based on a test segment corresponding to type I pelvic floor muscle fibers and a test segment corresponding to type II pelvic floor muscle fibers, respectively. The method includes determining a muscle strength grade and/or a fatigue degree for the type I muscle by combining the first baseline image and the test curve corresponding to the type I pelvic floor muscle fibers. The method includes determining a muscle strength grade and/or a fatigue degree for the type II muscle by combining the second baseline image and the test curve corresponding to the type II pelvic floor muscle fibers.

### CROSS-REFERENCE TO RELATED APPLICATIONS

This application claims priority to the Chinese Patent Application No. 202411933842.2, filed on Dec. 26, 2024, the contents of which are hereby incorporated by reference.

### TECHNICAL FIELD

The present disclosure relates to the field of pelvic floor muscle evaluation, and in particular, to a method, system, and apparatus for evaluating test data of pelvic floor muscle.

### BACKGROUND

Currently, pelvic floor muscle dysfunction is primarily determined based on a muscle strength of the pelvic floor muscle. Traditional screening for the pelvic floor muscle dysfunction collects a vaginal contraction pressure of a subject using a pressure balloon or collects a vaginal electromyography (EMG) value using an electrode probe for assessment. Data collected in a single session is singular, which is not conducive to accurate screening of the pelvic floor muscle dysfunction.

With a growth in demand for home-based screening and remote rehabilitation, automation of an evaluation process, consistency of results, and immediacy of feedback have become focal points in the field. In existing systems for screening the pelvic floor muscle dysfunction, when evaluating the test data of the pelvic floor muscle, there are still shortcomings of significant subjective human influence and low evaluation efficiency.

Therefore, there is desired to provide a method, system, and apparatus for evaluating test data of pelvic floor muscle, which may accurately and rapidly determine a muscle strength grade and a fatigue degree on pelvic floor muscle data, reduce subjective human influence, and help improve an accuracy and efficiency for evaluating the test data of the pelvic floor muscle.

### SUMMARY

One or more embodiments of the present disclosure provide a method for evaluating test data of pelvic floor muscle. The method includes following operations including: obtaining the test data of the pelvic floor muscle within a test period, wherein the test data includes an evaluation metric with temporal information, the evaluation metric includes an electromyography (EMG) value V and/or a pressure value P of vagina, and the test data includes a test segment corresponding to type I pelvic floor muscle fibers and a test segment corresponding to type II pelvic floor muscle fibers; presetting a threshold value Bou_val, obtaining a first baseline image for type I pelvic floor muscle fibers and a second baseline image for type II pelvic floor muscle fibers, constructing a test curve corresponding to type I pelvic floor muscle fibers and a test curve corresponding to type II pelvic floor muscle fibers, respectively, based on the test segment corresponding to the type I pelvic floor muscle fibers and the test segment corresponding to the type II pelvic floor muscle fibers; wherein a planar Cartesian coordinate system is constructed with a test time as an X-axis and a percentage obtained by dividing an evaluation metric in the test segment corresponding to the type I pelvic floor muscle fibers by a maximum value of an evaluation metric in the first baseline image as a Y-axis, and a line formed in the system is the test curve corresponding to the type I pelvic floor muscle fibers; a planar Cartesian coordinate system is constructed with the test time as an X-axis and a percentage obtained by dividing an evaluation metric in the test segment corresponding to the type II pelvic floor muscle fibers by a maximum value of an evaluation metric in the second baseline image as a Y-axis, and a line formed in the system is the test curve corresponding to the type II pelvic floor muscle fibers; determining a muscle strength grade and/or a fatigue degree for the type I muscle by combining the first baseline image and the test curve corresponding to the type I pelvic floor muscle fibers; and determining a muscle strength grade and/or a fatigue degree for the type II muscle by combining the second baseline image and the test curve corresponding to the type II pelvic floor muscle fibers; wherein the muscle strength grade for the type I muscle includes: designating a contraction area of the test curve corresponding to the type I pelvic floor muscle fibers as a first test area S_{A1}, designating an area of the first baseline image that does not exceed the threshold value Bouᵥₐₗ as a first reference area S_{B1}, and obtaining a result of the muscle strength grade for the type I muscle based on a first ratio F₁ of the first test area S_{A1} to the first reference area S_{B1}; the muscle strength grade for the type II muscle includes: designating a contraction area of the test curve corresponding to the type II pelvic floor muscle fibers as a first test area S_{A2}, designating an area of the second baseline image that does not exceed the threshold value Bouᵥₐₗ as a second reference area S_{B2}, and obtaining a result of the muscle strength grade for the type II muscle based on a second ratio F₂ of the second test area S_{A2} to the second reference area S_{B2}; discretizing the test curve corresponding to the type I pelvic floor muscle fibers into n₁ consecutive data points in the muscle strength grade for the type I muscle, converting the first baseline image as a trapezoid-approximated image, and determining the first ratio F₁ of the first test area S_{A1} to the first reference area S_{B1} by following equation (1):

$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \% \qquad ( 1 )$$

wherein k denotes an ordinal number of a data point, k∈[1, n₁], pointₖ denotes a Y-axis corresponding to a k-th data point, t denotes a test duration of the test curve corresponding to the type I pelvic floor muscle fibers, D₁ and D₂ denote an upper base and lower base of a trapezoidal image, respectively, h₁ denotes a height of the trapezoidal image, and h₁ is equal to the threshold value Bou_val; in the muscle strength grade for the type I muscle: in response to the first ratio F₁ ∈[0%, 10%], outputting the result of the muscle strength grade for the type I muscle as a grade 0; in response to the first ratio F₁∈(10%, 28%], outputting the result of the muscle strength grade for the type I muscle as a grade 1; in response to the first ratio F₁∈(28%, 46%], outputting the result of the muscle strength grade for the type I muscle as a grade 2; in response to the first ratio F₁∈(46%, 64%], outputting the result of the muscle strength grade for the type I muscle as a grade 3; in response to the first ratio F₁∈(64%, 82%], outputting the result of the muscle strength grade for the type I muscle as a grade 4; and in response to the first ratio F₁∈(82%, 100%], outputting the result of the muscle strength grade for the type I muscle as a grade 5.

One or more embodiments of the present disclosure provide a system for evaluating test data of pelvic floor muscle. The system includes: an input module, configured to obtain the test data of the pelvic floor muscle within a test period, wherein the test data includes an evaluation metric with temporal information, the evaluation metric includes an electromyography (EMG) value V and/or pressure value P of vagina, and the test data includes a test segment corresponding to type I pelvic floor muscle fibers and a test segment corresponding to type II pelvic floor muscle fibers; a curve construction, configured to preset a threshold value Bou_val, obtain a first baseline image for type I pelvic floor muscle fibers and a second baseline image for type II pelvic floor muscle fibers, construct a test curve corresponding to type I pelvic floor muscle fibers and a test curve corresponding to type II pelvic floor muscle fibers, respectively, based on the test segment corresponding to the type I pelvic floor muscle fibers and the test segment corresponding to the type II pelvic floor muscle fibers; wherein a planar Cartesian coordinate system is constructed with a test time as an X-axis and a percentage obtained by dividing an evaluation metric in the test segment corresponding to the type I pelvic floor muscle fibers by a maximum value of an evaluation metric in the first baseline image as a Y-axis, and a line formed in the system is the test curve corresponding to the type I pelvic floor muscle fibers; a planar Cartesian coordinate system is constructed with the test time as an X-axis and a percentage obtained by dividing an evaluation metric in the test segment corresponding to the type II pelvic floor muscle fibers by a maximum value of an evaluation metric in the second baseline image as a Y-axis, and a line formed in the system is the test curve corresponding to the type II pelvic floor muscle fibers; a calculation module, configured to determine a muscle strength grade and/or a fatigue degree for the type I muscle by combining the first baseline image and the test curve corresponding to the type I pelvic floor muscle fibers; and determine a muscle strength grade and/or a fatigue degree for the type II muscle by combining the second baseline image and the test curve corresponding to the type II pelvic floor muscle fibers; wherein the muscle strength grade for the type I muscle includes: designating a contraction area of the test curve corresponding to the type I pelvic floor muscle fibers as a first test area S_{A1}, designating an area of the first baseline image that does not exceed the threshold value Bouᵥₐₗ as a first reference area S_{B1}, and obtaining a result of the muscle strength grade for the type I muscle based on a first ratio F₁ of the first test area S_{A1} to the first reference area S_{B1}; the muscle strength grade for the type II muscle includes: designating a contraction area of the test curve corresponding to the type II pelvic floor muscle fibers as a first test area S_{A2}, designating an area of the second baseline image that does not exceed the threshold value Bouᵥₐₗ as a second reference area S_{B2}, and obtaining a result of the muscle strength grade for the type II muscle based on a second ratio F₂ of the second test area S_{A2} to the second reference area S_{B2}; discretizing the test curve corresponding to the type I pelvic floor muscle fibers into n₁ consecutive data points in the muscle strength grade for the type I muscle, converting the first baseline image as a trapezoid-approximated image, and determining the first ratio F₁ of the first test area S_{A1} to the first reference area S_{B1} by a following equation (1):

$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \% \qquad ( 1 )$$

wherein k denotes an ordinal number of a data point, k∈[1, n₁], pointₖ denotes a Y-axis corresponding to a k-th data point, t denotes a test duration of the test curve corresponding to the type I pelvic floor muscle fibers, D₁ and D₂ denote an upper base and lower base of a trapezoidal image, respectively, h₁ denotes a height of the trapezoidal image, and h₁ is equal to the threshold value Bou_val; in the muscle strength grade for the type I muscle: in response to the first ratio F₁∈[0%, 10%], outputting a result of the muscle strength grade for the type I muscle as a grade 0; in response to the first ratio F₁∈(10%, 28%], outputting a result of the muscle strength grade for the type I muscle as a grade 1; in response to the first ratio F₁∈(28%, 46%], outputting a result of the muscle strength grade for the type I muscle as a grade 2; in response to the first ratio F₁∈(46%, 64%], outputting a result of the muscle strength grade for the type I muscle as a grade 3; in response to the first ratio F₁∈(64%, 82%], outputting a result of the muscle strength grade for the type I muscle as a grade 4; and in response to the first ratio F₁∈(82%, 100%], outputting a result of the muscle strength grade for the type I muscle as a grade 5.

One or more embodiments of the present disclosure provide a computer apparatus. The computer apparatus includes a processor and a memory in signal communication. The memory stores at least one instruction or at least one program segment, and the processor, when loading the at least one instruction or the at least one program segment, is configured to execute the method for evaluating test data of pelvic floor muscle as described in any embodiments of the present disclosure.

One or more embodiments of the present disclosure provide a computer-readable storage medium. The computer-readable storage medium stores at least one instruction or at least one program segment. The at least one instruction or the at least one program segment, when loaded and executed by a processor, causes the processor to perform the method for evaluating test data of pelvic floor muscle as described in any embodiments of the present disclosure.

Beneficial effects of embodiments of the present disclosure include, but are not limited to:

A baseline image and a test curve are constructed based on test data of the pelvic floor muscle. Combined with features of force exertion of type I muscle fibers and type II muscle fibers, the threshold value is set. The muscle strength grade for the type I muscle is determined by using a ratio of a contraction area of the test curve corresponding to the type I pelvic floor muscle fibers to an area of a portion of the first baseline image that does not exceed the threshold value. The muscle strength grade for the type II muscle is determined by using a ratio of an area of a portion of the test curve corresponding to type II pelvic floor muscle fibers that exceeds the threshold value to an area of a portion of the second baseline image that exceeds the threshold value. Therefore, the muscle strength grades for the type I muscle fibers and the type II muscle fibers can be automatically calculated, the fatigue degree can be calculated based on the baseline image and the test curve, and manual observation and judgment are not required. The method can accurately and quickly determine the muscle strength grade and the fatigue degree on the pelvic floor muscle data, reduce subjective human influence, and help improve the accuracy and efficiency for evaluating the test data of the pelvic floor muscle.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a flowchart illustrating an exemplary process for evaluating test data of pelvic floor muscle according to some embodiments of the present disclosure;

FIG. 2 is a schematic diagram illustrating a first test area of a test curve corresponding to type I pelvic floor muscle fibers according to some embodiments of the present disclosure;

FIG. 3 is a schematic diagram illustrating a first reference area of a first baseline image according to some embodiments of the present disclosure;

FIG. 4 is a schematic diagram illustrating a second test area of a test curve corresponding to type II pelvic floor muscle fibers according to some embodiments of the present disclosure;

FIG. 5 is a schematic diagram illustrating a second reference area of a second baseline image according to some embodiments of the present disclosure; and

FIG. 6 is a schematic structural diagram illustrating a computer apparatus according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit” and/or “module” used herein are a way to distinguish between different components, elements, parts, sections, or assemblies at different levels. However, the terms may be replaced by other expressions if other words accomplish the same purpose.

As shown in the present disclosure and in the claims, unless the context clearly suggests an exception, the words “one,” “a,” “an,” “one kind,” and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

Flowcharts are used in the present disclosure to illustrate the operations performed by a system according to embodiments of the present disclosure, and the related descriptions are provided to aid in a better understanding of the method and/or system. It should be appreciated that the preceding or following operations are not necessarily performed in an exact sequence. Instead, steps can be processed in reverse order or simultaneously. Also, it is possible to add other operations to these processes or to remove a step or steps from these processes.

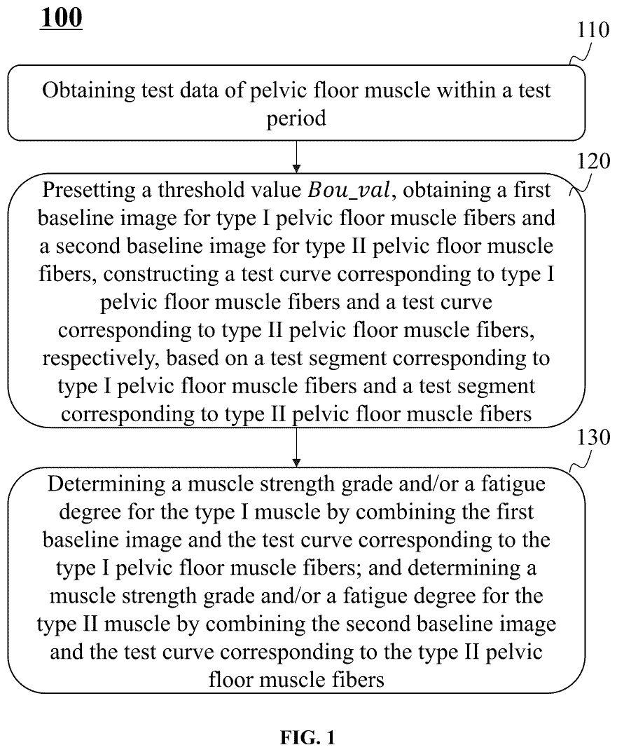

FIG. 1 is a flowchart illustrating an exemplary process for evaluating test data of pelvic floor muscle according to some embodiments of the present disclosure. In some embodiments, the method may be performed by a processor.

The processor may process data and/or information obtained from other devices or system components. The processor may execute program instructions based on such data, information, and/or processing results to perform one or more of the functions described herein. In some embodiments, the processor may include one or more sub-processing devices (e.g., a single-core processing device or a multi-core processing device). Merely by way of example, the processor may include a microcontrol unit (MCU), a central processing unit (CPU), a controller, a microprocessor, or the like, or any combination thereof. In some embodiments, the processor may include a plurality of modules, and different modules may be used to execute separate program instructions.

As shown in FIG. 1, a method for evaluating test data of pelvic floor muscle according to some embodiments of the present disclosure, i.e., a process 100, includes the following operations:

In 110, the test data of the pelvic floor muscle within a test period is obtained.

The test period refers to a time period corresponding to a complete contraction and relaxation cycle of the pelvic floor muscle.

The test data refers to data related to a pelvic floor muscle test.

In some embodiments, the test data includes an evaluation metric (EMG amplitude and/or intra-vaginal pressure value) with temporal information. The evaluation metric refers to a metric for evaluating the test data of the pelvic floor muscle. In some embodiments, the evaluation metric includes an electromyography (EMG) value V and/or a pressure value P of vagina.

In some embodiments, the processor may obtain the test data of the pelvic floor muscle within the test period through the pelvic floor muscle test. For example, a subject may be guided to perform pelvic floor muscle contraction and expansion actions, and the test data of the pelvic floor muscle of the subject within the test period may be collected. The test data includes the evaluation metric with the temporal information. The pelvic floor muscle test is a continuous process, therefore, the collected test data is a string of continuous data with the temporal information.

In some embodiments, the evaluation metric may be one of the EMG value and the pressure value, or simultaneously both the EMG value and the pressure value. In some embodiments, the EMG value may be measured by a surface EMG sensor (e.g., a pelvic floor muscle EMG probe), and the pressure value may be measured by a pressure sensor (e.g., the pelvic floor muscle pressure probe). In some embodiments, the EMG value and the pressure value may be synchronously collected by an integrated EMG and pressure probe (a pelvic floor muscle function comprehensive detection probe or a multi-source fusion probe may also be used). The collected EMG value and pressure value may be selected for use during a data evaluation stage based on a stage of the subject. For example, an appropriate evaluation metric is selected based on whether the subject is in a screening stage or a treatment effect evaluation stage.

In some embodiments, since muscle strength grades need to be determined for type I muscle fibers and type II muscle fibers, respectively, the test data includes a test segment corresponding to type I pelvic floor muscle fibers and a test segment corresponding to type II pelvic floor muscle fibers.

The test segment corresponding to type I pelvic floor muscle fibers and the test segment corresponding to type II pelvic floor muscle fibers refer to a segment of the test data where the type I muscle fibers account for a relatively high proportion and a segment of the test data where the type II muscle fibers account for a relatively high proportion in the test data, respectively. The type I muscle fibers refer to muscle fibers with a slow contraction speed, a small generated force, but extremely strong fatigue resistance, which are also known as slow-twitch muscle fibers. The type II muscle fibers refer to muscle fibers with a fast contraction speed, capable of generating strong force, but prone to fatigue, which are also known as fast-twitch muscle fibers. In some embodiments, the test segment corresponding to type I pelvic floor muscle fibers and the test segment corresponding to type II pelvic floor muscle fibers may include, but are not limited to, a sustained contraction segment, a rhythmic burst segment, a transition segment, or the like, or any combinations thereof.

In a test process, two segments of curves where the type I muscle fibers and the type II muscle fibers primarily work may be obtained, respectively, by standardizing a process of guiding the subject to exert force, and the two segments may be distinguished based on working features of the type I muscle fibers and the type II muscle fibers. Merely by way of example, the type I muscle fibers perform sustained and relatively small force exertion. A change curve of the EMG value and pressure value for the type I muscle fibers approximates a trapezoid, with a relatively flat curve segment in the middle, and part of the curve is below a threshold value. The type II muscle fibers perform intermittent and relatively large force exertion. A change curve of the EMG value and the pressure value for the type II muscle fibers approximates multiple discontinuous peaks, and part of the curve is above the threshold value. Therefore, by obtaining the test data of the subject within the test period, the test segment corresponding to type I pelvic floor muscle fibers and the test segment corresponding to type II pelvic floor muscle fibers may be obtained, respectively.

In some embodiments, the processor may further be configured to: obtain the test data within a plurality of collection periods. In each collection period, the processor performs: extracting an action quality feature from the test data of the collection period; judging whether the test data is valid data based on the action quality feature and physiological information of a user through a first vector database; in response to a determination that the test data is the valid data, proceeding to a next collection period of the collection period; in response to a determination that the test data is not valid data, determining a non-compliance reason based on the action quality feature, and generating a prompt instruction based on the non-compliance reason. The prompt instruction is configured to control a prompt device to play the prompt instruction to guide the user to complete a standard action in a next collection period.

The collection period refers to a period corresponding to one data collection. In one collection period, the user may be guided to perform multiple actions. For example, the user may be guided to perform 5 burst contractions, i.e., 5 test periods.

The action quality feature refers to a metric that quantifies a pelvic floor muscle action. In some embodiments, the action quality feature may include a type I action quality feature related to the type I muscle fibers and a type II action quality feature related to the type II muscle fibers.

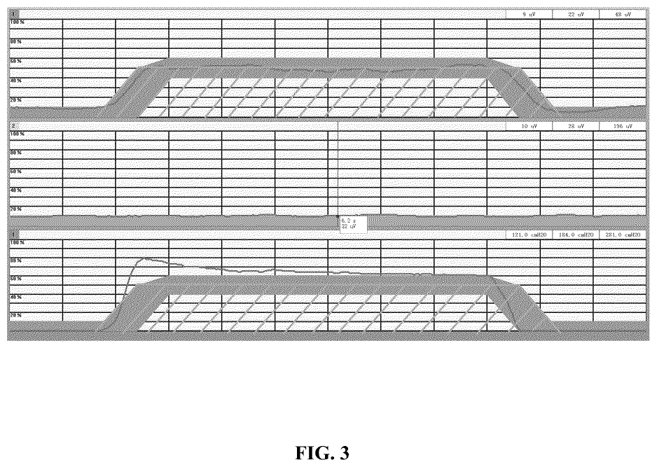

In some embodiments, the type I action quality feature may include metrics such as a recruitment speed, a signal peak value, and a maintenance stability, or the like, in a test curve corresponding to type I pelvic floor muscle fibers. The recruitment speed refers to a slope of a rising segment of the EMG value or the pressure value constituted by the evaluation metric in the test segment corresponding to type I pelvic floor muscle fibers, as a rising segment shown in FIG. 3. The signal peak value refers to a signal amplitude at a peak point in the test segment corresponding to the type I pelvic floor muscle fibers, i.e., a maximum signal amplitude. The maintenance stability refers to a signal standard deviation of a steady segment (as shown in FIG. 3) in the test segment corresponding to the type I pelvic floor muscle fibers.

In some embodiments, the type II action quality feature may include metrics such as a signal peak value, a reaction speed, a relaxation speed, or the like, in a test curve corresponding to type II pelvic floor muscle fibers. The signal peak value refers to an average value of signal amplitudes of a plurality of peak points in the test segment corresponding to type II pelvic floor muscle fibers. The reaction speed refers to a time from when an instruction is issued until a signal first reaches a peak in the test segment corresponding to type II pelvic floor muscle fibers. The relaxation speed refers to an average value of times for the signal to return to a baseline level after each contraction in the test segment corresponding to type II pelvic floor muscle fibers. The baseline level refers to a straight-line segment portion in a second baseline image.

More descriptions regarding the test curve corresponding to type I pelvic floor muscle fibers, the test curve corresponding to type II pelvic floor muscle fibers, and the second baseline image may be found in the related descriptions below.

In some embodiments, the action quality features may be obtained by statistics of evaluation metrics of the temporal information.

The physiological information refers to basic biomedical data of the user. In some embodiments, the physiological information of the user includes an age, a height, a weight, or the like, of the user.

The valid data refers to test data for which a user action meets a standard.

In some embodiments, the processor may determine preset thresholds for a plurality of metrics in the action quality feature based on the physiological information of the user via a first vector database. The processor may construct the physiological information of the user as a first vector, obtain the preset thresholds for the plurality of metrics in the action quality feature by querying the first vector database based on the first vector. The preset thresholds for the plurality of metrics in the action quality feature are used to determine whether the test data is the valid data.

The first vector database includes a plurality of reference first vectors and

corresponding labels. The reference first vector is constructed similarly to the first vector. The plurality of reference first vectors are constructed from historical physiological information of a plurality of historical users. The label corresponding to the reference first vector is an optimal threshold for the plurality of metrics in the action quality feature corresponding to the vector. For each metric, the processor determines a historical threshold among a plurality of historical thresholds used by the plurality of historical users corresponding to the reference first vector, where a subsequently obtained muscle strength grade and fatigue degree are closest to manually annotated muscle strength grade and fatigue degree as the label corresponding to the feature vector.

In some embodiments, the processor selects a reference first vector with the highest similarity to the first vector from the first vector database and corresponding label as the preset threshold for the plurality of metrics in the action quality features corresponding to the first vector.

In some embodiments, the processor determines whether all metrics in the action quality feature extracted from the test data satisfy corresponding preset thresholds (including being greater than or less than the corresponding preset thresholds). In response to a determination that all metrics in the action quality feature satisfy the corresponding preset thresholds, the processor determines that the test data is the valid data, otherwise, the processor determines that the test data is not the valid data.

The non-compliance reason refers to a reason why the test data is not the valid data. For example, the non-compliance reason may be “insufficient strength”, “relaxing too slowly”, or the like. The non-compliance reason includes one or more reasons.

In some embodiments, the processor determines the non-compliance reason based on metrics that do not satisfy the corresponding preset thresholds. For example, in response to a determination that the relaxation speed is less than a corresponding relaxation speed threshold, it indicates that the user relaxes too slowly, and the corresponding non-compliance reason is relaxing too slowly.

The prompt instruction refers to an instruction for prompting an amplitude, a speed, or a frequency of an action performed by the user. The prompt instruction includes one or more prompt contents. For example, the prompt content may be “a little more strength is needed, please use more force next time” or “relax more thoroughly”, or the like.

In some embodiments, the processor obtains the prompt content corresponding to the non-compliance reason by accessing a first preset table. The first preset table includes the non-compliance reason and corresponding prompt content. The first preset table is set based on experience.

In some embodiments of the present disclosure, by determining action quality in real time during a collection process and providing targeted prompts, it is ensured that the test data finally used for evaluation is standard and valid, thereby improving the accuracy and reliability of an evaluation result.

In 120, a threshold value Bou_val is preset, a first baseline image for type I pelvic floor muscle fibers and a second baseline image for type II pelvic floor muscle fibers are obtained, the test curve corresponding to type I pelvic floor muscle fibers and the test curve corresponding to type II pelvic floor muscle fibers are constructed respectively based on the test segment corresponding to the type I pelvic floor muscle fibers and the test segment corresponding to the type II pelvic floor muscle fibers.

The threshold value Bou_val refers to a threshold related to division of a test curve for the pelvic floor muscle. In some embodiments, the threshold value Bou_val includes, but is not limited to, a proportional value, a fixed value, a dynamically calculated value, or the like, or any combination thereof. In some embodiments, the threshold value Bou_val is preset manually based on experience.

In some embodiments, the threshold value Bou_val is 50%. In some embodiments, the threshold value Bou_val is one of 40%, 45%, 50%, 55%, 60%, or the like.

The first baseline image refers to a reference image for the pelvic floor muscle type I muscle fibers. The second baseline image refers to a reference image for the pelvic floor muscle type II muscle fiber.

The test curve corresponding to type I pelvic floor muscle fibers refers to a curve corresponding to the test segment corresponding to the type I pelvic floor muscle fibers obtained from the test data. The test curve corresponding to type II pelvic floor muscle fibers refers to a curve corresponding to the test segment corresponding to the type II pelvic floor muscle fibers obtained from the test data.

In some embodiments, the processor constructs a planar Cartesian coordinate system with a test time as an X-axis and a percentage obtained by dividing an evaluation metric (e.g., the EMG value V and/or the pressure value P) in the test segment corresponding to the type I pelvic floor muscle fibers in the test data by a maximum value of the evaluation metric in the first baseline image as a Y-axis. A line formed in the system is the test curve corresponding to the type I pelvic floor muscle fibers. The processor constructs the planar Cartesian coordinate system with the test time as an X-axis and a percentage obtained by dividing an evaluation metric in the test segment corresponding to the type II pelvic floor muscle fibers in the test data by a maximum value of the evaluation metric in the second baseline image as a Y-axis. A line formed in the system is the test curve corresponding to the type II pelvic floor muscle fibers.

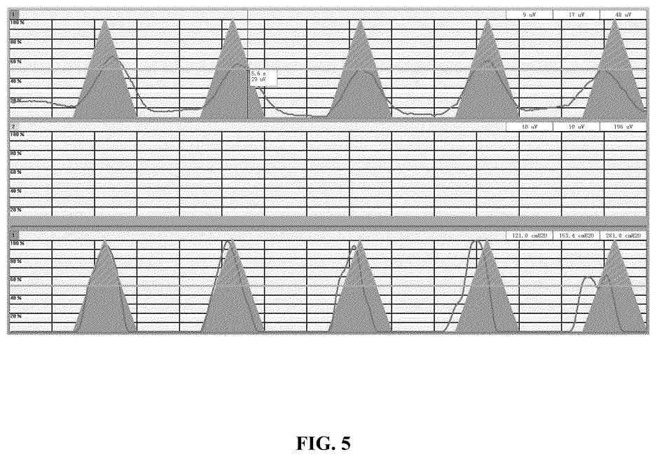

FIG. 3 is a schematic diagram illustrating a first reference area of a first baseline image according to some embodiments of the present disclosure. FIG. 5 is a schematic diagram illustrating a second reference area of a second baseline image according to some embodiments of the present disclosure. More descriptions regarding the first reference area and the second reference area may be found in the related descriptions below.

In some embodiments, the first baseline image and the second baseline image are constructed with reference to an internationally common pelvic floor muscle strength grade test manner. For example, with reference to the internationally common pelvic floor muscle strength grade test manner, combined with contraction features of the type I muscle and contraction features of the type II muscle, using a maximum contraction pressure/EMG value of the vagina as a highest value of the Y-axis, and further combined with the set threshold value Bou_val, the first baseline image and the second baseline image are constructed, respectively. As shown in FIG. 3 and FIG. 5, the first baseline image is in a trapezoid-like shape, and the second baseline image includes a plurality of intermittently distributed triangles. The baseline images serve as reference baselines for pelvic floor muscle strength grading. A straight-line segment in the first baseline image and the second baseline image is the threshold value. The type I muscle fibers have contraction features including resistance to fatigue and long endurance duration. The type II muscle fibers have contraction features including a strong contraction force and fast contraction.

The muscle strength grade refers to grading a contraction intensity of the pelvic floor muscle. In some embodiments, a result of muscle strength grade may include, but is not limited to, a grade 0, a grade 1, a grade 2, a grade 3, a grade 4, a grade 5, or the like, or any combination thereof.

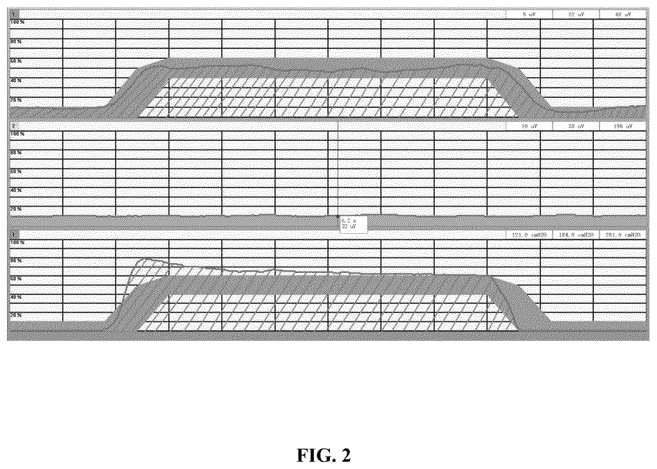

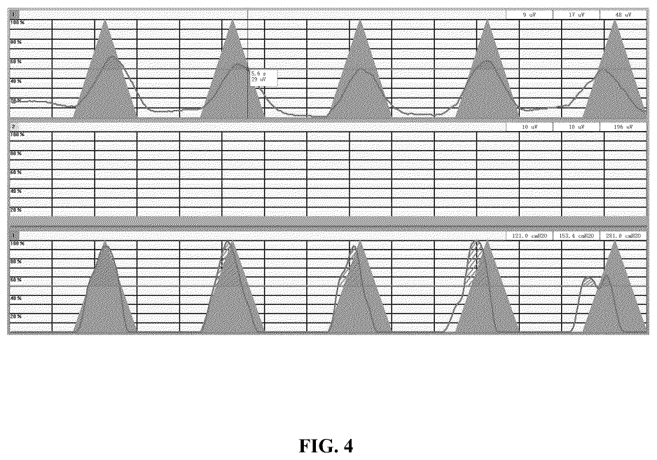

FIG. 2 is a schematic diagram illustrating a first test area of a test curve corresponding to type I pelvic floor muscle fibers according to some embodiments of the present disclosure. FIG. 4 is a schematic diagram illustrating a second test area of a test curve corresponding to type II pelvic floor muscle fibers according to some embodiments of the present disclosure. More descriptions regarding the first test area and the second test area may be found in the related descriptions below.

Based on the test segment corresponding to the type I pelvic floor muscle fibers, the test segment corresponding to the type I pelvic floor muscle fibers is fused with the obtained first baseline image to construct the test curve corresponding to the type I pelvic floor muscle fibers, thereby obtaining a test image as shown in FIG. 2. In some embodiments, a planar Cartesian coordinate system is constructed with a test time as an X-axis and a percentage obtained by dividing an evaluation metric (e.g., the pressure value P) in the test segment corresponding to the type I pelvic floor muscle fibers by a maximum pressure value in the first baseline image as a Y-axis. A line formed is the test curve corresponding to the type I pelvic floor muscle fibers. Similarly, the test curve corresponding to the type II pelvic floor muscle fibers may be obtained, i.e., a test image as shown in FIG. 4.

In some embodiments, to obtain the first baseline image for the type I pelvic floor muscle fibers and the second baseline image for the type II pelvic floor muscle fibers, the processor may further generate the first baseline image and the second baseline image based on the physiological information, gynecological and obstetric information, and habit information of the user by a baseline construction model.

The gynecological and obstetric information refers to medical information related to a female reproductive system, pregnancy, childbirth, and postpartum recovery. In some embodiments, the gynecological and obstetric information may include a count of pregnancies, a count of deliveries, a delivery mode (e.g., a vaginal delivery or a cesarean section), presence or absence of instrumental delivery, a neonatal weight, or the like.

The habit information refers to daily behavior or exercise information of the user. In some embodiments, the habit information may include an exercise frequency, a daily sedentary duration, presence or absence of factors increasing abdominal pressure, such as chronic cough/constipation, or the like.

In some embodiments, the processor may obtain the physiological information, the gynecological and obstetric information, and the habit information of the user directly via input by a staff.

In some embodiments, the baseline construction model is a machine learning model. For example, the baseline construction model includes a neural network (NN) model, a recurrent neural network (RNN) model, or the like.

In some embodiments, inputs of the baseline construction model may include the physiological information, the gynecological and obstetric information, and the habit information of the user, outputs of the baseline construction model may include the first baseline image and the second baseline image.

In some embodiments, the baseline construction model may be obtained by training based on training data. In some embodiments, the processor may obtain a plurality of first training samples with first labels to constitute a first training sample set, and perform a plurality of rounds of iterations based on the first training sample set.

In some embodiments, the processor may input the first training sample set into an initial baseline construction model to perform the plurality of rounds of iterations. Each round of iteration includes: selecting one or more first training samples from the first training sample set; inputting the one or more first training samples into the initial baseline construction model; obtaining one or more model estimation output corresponding to the one or more first training samples; substituting the one or more model estimation outputs corresponding to the one or more first training samples and the first labels of the one or more first training samples into a equation of a predefined loss function to calculate a value of the loss function; and inversely updating model parameters of the initial baseline construction model based on the value of the loss function. When an iteration end condition is satisfied, the iteration ends to obtain the trained baseline construction model. The iteration end condition may be that the loss function converges, a count of iterations reaching a threshold, or the like.

The first training sample may include historical physiological information, historical gynecological and obstetric information, and historical habit information of the user. The first label may be an optimal first baseline image and an optimal second baseline image corresponding to the first training sample. The first training sample set may include first training samples corresponding to a plurality of different users.

When determining the first label, the processor may determine, from a plurality of different first historical baseline images and second historical baseline images corresponding to the first training sample, a first historical baseline image and a second historical baseline image having a highest morphological similarity to an actual test curve of the user as the optimal first baseline image and the optimal second baseline image, and then as the first label corresponding to the first training sample. The morphological similarity refers to a similarity of a curve trend.

The actual test curve refers to a curve with a best comprehensive performance among a plurality of actual test curves of a historical user after recovery (i.e., after a pelvic floor muscle function returns to normal).

A condition after recovery may include at least one of: a result of the muscle strength grade for the type I muscle and a result of the muscle strength grade for the type II muscle remain stable at a grade 4 or above; a fatigue resistance for the type I muscle fibers and a fatigue resistance for the type II muscle fibers are qualified; symptoms of pelvic floor muscle dysfunction (e.g., stress urinary incontinence, pelvic organ prolapse, etc.) of the user are cured.

In some embodiments, the qualified fatigue resistance for the type I muscle fibers may be manifested as: being able to easily complete a sustained contraction of 8-10 seconds, with a very low decay rate (e.g., a force decline does not exceed 10-15% of an initial peak value during an entire plateau maintenance period) during a plateau maintenance period (i.e., a steady segment); a result of fatigue degree calculation is stably “low fatigue” or “no significant fatigue”. In some embodiments, the qualified fatigue resistance for the type II muscle fibers may be manifested as: being able to continuously complete 8-12 standard explosive contractions, with no significant decrease in peak height; a baseline between contractions shows no significant elevation, indicating that the muscle is capable of relaxing and recovering quickly and thoroughly.

In some embodiments, for the test curve corresponding to the type I pelvic floor muscle fibers, the best comprehensive performance includes a highest weighted sum obtained by performing normalization processing on a first strength metric, an endurance metric, and a control metric and then performing weighted summation. The first strength metric includes a contraction area and an average amplitude during the plateau maintenance period. The endurance metric refers to a duration of the plateau maintenance period. The control metric refers to a time of a rising segment and a time of a falling segment. The normalization processing includes Min-Max normalization, etc. Weight coefficients for the weighted summation are set empirically. A weight coefficient for the control metric is a negative number.

In some embodiments, for the test curve corresponding to the type II pelvic floor muscle fibers, the best comprehensive performance includes a highest weighted sum obtained by performing the normalization processing on a second strength metric, a reaction metric, and a fatigue resistance metric and then performing weighted summation. The second strength metric refers to an average signal amplitude and a maximum signal amplitude of a plurality of peak points. The reaction metric refers to an average reaction time from an instruction issuance to a signal start rising, an average rise time from the start rising to reaching a peak, and an average relaxation time from the peak falling to completely returning to a baseline. The fatigue resistance metric refers to a standard deviation of signal amplitudes of the plurality of peak points. The normalization processing includes Min-Max normalization, etc. Weight coefficients for the weighted summation are set empirically. Weight coefficients for the reaction metric and the fatigue resistance metric are negative numbers.

In some embodiments of the present disclosure, generating a personalized baseline image by the baseline construction model based on the physiological information of the user improves personalized evaluation accuracy and a clinical reference value.

In some embodiments, the processor may further perform following operations at a preset cycle: determining a type I morphological parameter and a type II morphological parameter of the user based on a plurality of historical test data; determining a first evaluation image and a second evaluation image based on the type I morphological parameter and the type II morphological parameter; updating the first baseline image based on the first evaluation image; and updating the second baseline image based on the second evaluation image.

The preset cycle refers to a preset count of evaluation. For example, the preset cycle may be 2 times, 3 times, 5 times, 10 times, etc.

The historical test data refers to test data of the user within the preset cycle.

The type I morphological parameter refers to a key morphological parameter in the test segment corresponding to the type I pelvic floor muscle fibers in the test data. In some embodiments, the type I morphological parameter includes the contraction area, an upper base, a lower base, and a height of a trapezoid.

The type II morphological parameter refers to a key morphological parameter in a test segment corresponding to type II pelvic floor muscle fibers in test data. In some embodiments, the type II morphological parameter includes a count of peaks, an average peak height, and an average peak interval.

In some embodiments, the processor is configured to calculate, for each historical test data, the contraction area, the upper base, the lower base, and the height of the trapezoid in the test segment corresponding to the type I pelvic floor muscle fibers, and the count of peaks, the average peak height, and the average peak interval in the test segment corresponding to type II pelvic floor muscle fibers. The processor is configured to determine an average contraction area, an average upper base, an average lower base, and an average height of the trapezoid in the test segment corresponding to the type I pelvic floor muscle fibers from the plurality of historical test data as the type I morphological parameter. The processor is configured to determine an average count of peaks, an average of the average peak height, and an average of the average peak interval in the test segment corresponding to the type II pelvic floor muscle fibers from the plurality of historical test data as the type II morphological parameter.

The first evaluation image refers to an evaluation curve for the type I muscle generated based on the type I morphological parameter.

The second evaluation image refers to an evaluation curve for the type II muscle generated based on the type II morphological parameter.

A difference between an evaluation curve and a test curve is that a Y-axis of the test curve is a ratio of an actual EMG value or an actual pressure value to a maximum value of a baseline image, and a Y-axis of the evaluation curve is the actual EMG value or the actual pressure value itself. That is, the Y-axis of the test curve corresponding to the type I pelvic floor muscle fibers is the actual EMG value or the actual pressure value corresponding to the type I morphological parameter, and the X-axis is the test time. The Y-axis of the test curve corresponding to the type II pelvic floor muscle fibers is the actual EMG value or the actual pressure value corresponding to the type II morphological parameter, and the X-axis is the test time.

In some embodiments, the processor is configured to perform a weighted summation of the first evaluation image and the first baseline image to update the first baseline image. The processor is configured to perform a weighted summation of the second evaluation image and the second baseline image to update the second baseline image.

In some embodiments, the processor is configured to perform a weighted summation of a Y-axis of the first evaluation image and a corresponding Y-axis of the first baseline image to update the Y-axis of the first baseline image. The processor is configured to perform a weighted summation of a Y-axis of the second evaluation image and a corresponding Y-axis of the second baseline image to update the Y-axis of the second baseline image.

In some embodiments, a weight of the first evaluation image and a weight of the second evaluation image are preset based on experience.

In some embodiments, the weight of the first evaluation image and the weight of the second evaluation image are proportional to a count of treatment sessions of the user. The count of treatment sessions of the user refers to a count of times the user undergoes treatment.

In some embodiments of the present disclosure, by increasing a weight of historical performance as the count of treatment sessions increases, a dynamically updated baseline image is enabled to focus more on a recent true ability of the user, thereby making an evaluation target more realistic.

In some embodiments of the present disclosure, by periodically updating a personalized baseline image of the user based on historical test performance of the user, dynamic self-adaptation of an evaluation standard is achieved, which can more sensitively track and reflect a rehabilitation progress of the user. A state of the pelvic floor muscle of the user is not constant during a recovery process. For example, immediately after childbirth, the state of the pelvic floor muscle is very poor. The poor state is predictable, and a baseline lower than that of a normal person may be set at this time. As treatment and repair progress, the state of the pelvic floor muscle gradually improves, and a corresponding baseline also becomes higher. Treatment is adjusted based on an actual image and the baseline image at each stage, so that the actual image continuously approaches the baseline image at a current stage. A final achieved effect is that the final actual image and the baseline image are substantially consistent. Therefore, during a recovery period of the user, i.e., a treatment course, the optimal first baseline image and the optimal second baseline image corresponding to the user are dynamically adjusted according to a recovery condition of the user, thereby making the first baseline image and the second baseline image closer to an actual state of the user.

In 130, a muscle strength grade and/or a fatigue degree for the type I muscle is determined by combining the first baseline image and the test curve corresponding to the type I pelvic floor muscle fibers, and a muscle strength grade and/or a fatigue degree for the type II muscle is determined by combining the second baseline image and the test curve corresponding to the type II pelvic floor muscle fibers.

The muscle strength grade for the type I muscle refers to grading of a contraction intensity of the type I muscle.

In some embodiments, a process for the muscle strength grade for the type I muscle includes: designating a contraction area of the test curve corresponding to the type I pelvic floor muscle fibers as a first test area S_{A1}, designating an area of the first baseline image that does not exceed the threshold value Bou_val as a first reference area S_{B1}, and obtaining a result of the muscle strength grade for the type I muscle based on a first ratio F₁ of the first test area S_{A1} to the first reference area S_{B1}.

For example, the contraction area of the test curve corresponding to the type I pelvic floor muscle fibers is an area of a region with blue left-slanting lines as shown in FIG. 2, i.e., an area enclosed by the test curve corresponding to the type I pelvic floor muscle fibers and the X-axis, and the area is recorded as the first test area S_{A1}.

For example, as shown in FIG. 3, an area enclosed by a portion of the first baseline image below the threshold value and the X-axis is taken as the first reference area S_{B1}, which is approximately represented as an area of a region with green left-slanting lines in FIG. 3.

In some embodiments, in the muscle strength grade for the type I muscle, the test curve corresponding to type I pelvic floor muscle fibers is discretized into n₁ consecutive data points. The first baseline image is converted into a trapezoid-approximated image. Based on an X-axis and a Y-axis corresponding to a plurality of data points of the test curve corresponding to the type I pelvic floor muscle fibers and an upper base, a lower base, and a height of a trapezoidal image, the first ratio F₁ of the first test area S_{A1} to the first reference area S_{B1} is determined according to a first ratio equation. The n₁ is an integer greater than 0.

For example, the first ratio equation may be a following equation (1):

$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \% \qquad ( 1 )$$

where, k denotes an ordinal number of a data point, k∈[1, n₁], pointₖ denotes a Y-axis corresponding to a k-th data point, t denotes a test duration of the test curve corresponding to the type I pelvic floor muscle fibers, D₁ and D₂ denote the upper base and the lower base of the trapezoidal image, respectively, h₁ denotes the height of the trapezoidal image, and h₁ is equal to the threshold value Bou_val. The n₁ is a quantity of the consecutive data points into which the test curve corresponding to the type I pelvic floor muscle fibers is divided.

Merely by way of example, the test curve corresponding to the type I pelvic floor muscle fibers is discretized into 400 consecutive data points. The upper base and the lower base of the trapezoidal image are 7.2 and 8.7, respectively. The test duration is 12s. The threshold value Bou_val is 50%. The first ratio F₁ may then be calculated as follows:

$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \%$$

In some embodiments, the processor is configured to determine the muscle strength grade for the type I muscle based on the calculated first ratio F₁: in response to the first ratio F₁∈[0%, 10%], output the result of the muscle strength grade for the type I muscle as a grade 0; in response to the first ratio F₁∈(10%, 28%], output the result of the muscle strength grade for the type I muscle as a grade 1; in response to the first ratio F₁∈(28%, 46%], output the result of the muscle strength grade for the type I muscle as a grade 2; in response to the first ratio F₁∈(46%, 64%], output the result of the muscle strength grade for the type I muscle as a grade 3; in response to the first ratio F₁∈(64%, 82%], output the result of the muscle strength grade for the type I muscle as a grade 4; and in response to the first ratio F₁∈(82%, 100%], output the result of the muscle strength grade for the type I muscle as a grade 5.

In some embodiments of the present disclosure, the first ratio is accurately calculated by above manner, reducing subjective human influence, thereby accurately determining the muscle strength grade for the type I muscle, which helps improve accuracy and efficiency of evaluating the test data of the pelvic floor muscle.

The determination of the fatigue degree refers to a quantitative evaluation of a rate and a degree of decline in functional output, such as strength or power, of a muscle fiber during sustained or repeated contractions.

In some embodiments, determining the fatigue degree for the type I muscle includes: selecting a first calculation point and a second calculation point in the test curve corresponding to the type I pelvic floor muscle fibers; and calculating the fatigue degree for the test curve corresponding to the type I pelvic floor muscle fibers using a fatigue degree calculation equation based on the first calculation point and the second calculation point. The T fatigue degree calculation equation may include an EMG fatigue degree equation and a pressure fatigue degree equation.

In some embodiments, a first peak point that exceeds a screening threshold in an initial segment of the test curve corresponding to the type I pelvic floor muscle fibers is selected and recorded as a first calculation point N₁. Another peak point after a certain time period following the first calculation point N₁ is selected as a second calculation point N₂. A first time point t₁ corresponding to the first calculation point N₁ and a second time point t₂ corresponding to the second calculation point N₂ are obtained. The initial segment refers to a portion of a curve from a first clear deviation from a baseline to an initial inflection point of a first negative-phase peak.

In some embodiments, in response to the evaluation metric including the EMG value V of the vagina, a fatigue degree B₁ for the test curve corresponding to the type I pelvic floor muscle fibers is calculated using the EMG fatigue degree equation based on the EMG value and the first time point t₁ corresponding to the first calculation point N₁, and the EMG value and the second time point t₂ corresponding to the second calculation point N₂.

For example, the EMG fatigue degree equation may be a following equation (2):

$${B}_{1} = - 100 \% * \{ \vert {P}_{1} - {P}_{2} \vert \div {P}_{1} ] \div \vert {t}_{2} - {t}_{1} \vert \} \qquad ( 2 )$$

where, V₁ denotes the EMG value corresponding to the first calculation point N₁, and V₂ denotes the EMG value corresponding to the second calculation point N₂.

In some embodiments, in response to the evaluation metric including the pressure value P of the vagina, the fatigue degree B₁ for the test curve corresponding to type I pelvic floor muscle fibers is calculated using the pressure fatigue degree equation based on the pressure value and the first time point t₁ corresponding to the first calculation point N₁, and the pressure value and the second time point t₂ corresponding to the second calculation point N₂.

For example, the pressure fatigue degree equation may be a following equation (3):

$${B}_{1} = - 100 \% * \{ \vert {P}_{1} - {P}_{2} \vert \div {P}_{1} ] \div \vert {t}_{2} - {t}_{1} \vert \} \qquad ( 3 )$$

where P₁ denotes a pressure value corresponding to the first calculation point N₁, and P₂ denotes a pressure value corresponding to the second calculation point N₂.

Merely by way of example, if the test duration is 12s, then according to steps of a contraction test, a first peak point exceeding 40% within the first 2.4s is taken as the first calculation point N₁. Simultaneously, a point 6s after the first peak point is taken as the second calculation point N₂. Time points and evaluation metrics corresponding to the first calculation point N₁ and the second calculation point N₂ are obtained, respectively, and a slope is calculated to characterize the fatigue degree for the type I muscle fibers.

In some embodiments of the present disclosure, the fatigue degree for the type I muscle fibers is accurately determined by the above manner without manual subjective judgment, thereby reducing the subjective human influence and helping to improve the accuracy and efficiency of evaluating the test data of the pelvic floor muscle.

The muscle strength grade for the type II muscle refers to grading of a contraction intensity of the type II muscle.

In some embodiments, a process for the muscle strength grade for the type II muscle includes: designating a contraction area of the test curve corresponding to the type II pelvic floor muscle fibers as a first test area S_{A2}, designating an area of the second baseline image that does not exceed the threshold value Bou_val as a second reference area S_{B2}, and obtaining a result of the muscle strength grade for the type II muscle based on a second ratio F₂ of the second test area S_{A2} to the second reference area S_{B2}.

For example, as shown in FIG. 4, an area of a portion of the test curve corresponding to the type II pelvic floor muscle fibers that exceeds the threshold value is taken as the second test area S_{A2}, i.e., an area of a region with blue left-slanting lines in FIG. 4.

For example, as shown in FIG. 5, an area of a portion of the second baseline image that exceeds the threshold value, i.e., an area of an upper part of a triangle, which is also an area of region with green left-slanting lines in FIG. 5, is taken as the second reference area S_{B2}.

In some embodiments, in the muscle strength grade for the type II muscle, a test duration of the test curve corresponding to the type II pelvic floor muscle fibers is evenly divided into m consecutive data intervals. Each data interval includes n₂ consecutive data points. A maximum baseline EMG value V_ref_max, a minimum baseline EMG value V_ref_min, a maximum baseline pressure value P_ref_max, and a minimum baseline pressure value P_ref_min of the second baseline image are obtained. An interval ratio F′ of the second test area S_{A2} to the second reference area S_{B2} in each data interval is calculated. The interval ratios F′ of the second test area S_{A2} to the second reference area S_{B2} of each data interval are summed and a mean value of the sum are calculated to obtain a second ratio F₂. The n₂ is an integer greater than 0.

In some embodiments, for each data interval, the processor may calculate the interval ratio F′ for the data interval using an interval ratio equation based on the second test area S_{A2} and the second reference area S_{B2} of the data interval.

For example, the interval ratio equation may be a following equation (4) and equation (5):

$${F}^{′} = \frac{{S}_{A 2}}{{S}_{B 2}} = \frac{{\sum }_{k = i}^{i + {n}_{2}} {pro}_{k}}{\frac{\frac{l}{T} * {n}_{2} * {h}_{2}}{2}} * 100 \% \qquad ( 4 )$$

$${pro}_{k} = \frac{V\_k - V\_ref \_min}{V\_ref \_max - V\_ref \_min} * 100 \% or \frac{P\_k - P\_ref \_min}{P\_ref \_max - P\_ref \_min} * 100 \% \qquad ( 5 )$$

where, i denotes a number of a first data point in the data interval, k denotes the ordinal number of the data point, k∈[1, m*n₂], V_k denotes an EMG value corresponding to the k-th data point, P_k denotes a pressure value corresponding to the k-th data point, l denotes a base length of a portion of the second baseline image where values exceeds the threshold value Bou_val, T denotes a time length of each data interval, and h₂ denotes a height of a portion of the second baseline image where values exceed the threshold value Bou_val. The n₂ is a quantity of consecutive data points in the each data interval.

Merely by way of example, the test duration is 15s. The 15s is evenly divided into 5 data intervals, which are 1-3 seconds, 4-6 seconds, 7-9 seconds, 10-12 seconds, and 13-15 seconds, respectively. A length of each data interval is 3 seconds. The base length of the portion of the second baseline image where values exceed the threshold value Bou_val is 0.8. Each data interval includes 80 consecutive data points, totaling 400 data points. Then, an interval ratio F′ of the second test area S_{A2} to the second reference area S_{B2} in each data interval is:

$${F}^{′} = \frac{{S}_{A 2}}{{S}_{B 2}} = \frac{{\sum }_{k = i}^{i + 80} {pro}_{k}}{\frac{\frac{0.8}{3} * 80 * 50}{2}} * 100 \%$$

Since there are 5 data intervals in total, resulting interval ratios may be F′₁, F′₂, F′₃, F′₄, and F′₅, respectively. A mean value then is calculated to determine the second ratio F₂:

$${F}_{2} = \frac{{F}_{1}^{′} + {F}_{2}^{′} + {F}_{3}^{′} + {F}_{4}^{′} + {F}_{5}^{′}}{5}$$

In some embodiments, the processor may determine the muscle strength grade for the type II muscle based on the calculated second ratio F₂: in response to the second ratio F₂∈(0%, 20%], outputting the result of the muscle strength grade for the type II muscle as a grade 1; in response to the second ratio F₂∈(20%, 40%], outputting the result of the muscle strength grade for the type II muscle as a grade 2; in response to the second ratio F₂∈(40%, 60%], outputting the result of the muscle strength grade for the type II muscle as a grade 3; in response to the second ratio F₂ € (60%, 80%], outputting the result of the muscle strength grade for the type II muscle as a grade 4; and in response to the second ratio F₂∈(80%, 100%], outputting the result of the muscle strength grade for the type II muscle as a grade 5.

In some embodiments of the present disclosure, the second ratio is accurately determined by the above manner, reducing the subjective human influence, thereby determining the muscle strength grade for the type II muscle, which helps improve the accuracy and efficiency of evaluating the test data of the pelvic floor muscle.

In some embodiments, before the muscle strength grade for the type II muscle, the processor may also: preset an EMG threshold V_thr of the EMG value V, and a contraction pressure threshold ΔP_thr of an active contraction pressure value ΔP; obtain a maximum value V_max of the EMG value V and a peak contraction pressure ΔP_max of the pressure value P in the test segment corresponding to the type II pelvic floor muscle fibers; in response to a grade condition for the type II muscle, perform the muscle strength grade for the type II muscle; the grade condition for the type II muscle including V_max>V_thr, ΔP_max>ΔP_thr, and at least one point in the test curve corresponding to the type II pelvic floor muscle fibers exceeding the threshold value Bou_val; and in response to the grade condition for the type II muscle, output the result of the muscle strength grade for the type II muscle as a grade 0.

In some embodiments, the processor may define the muscle strength grade for the type II muscle by combining work features of the type I muscle fibers and the type II muscle fibers, and preset the EMG threshold Vₜₕᵣ for the EMG value V and a contraction pressure threshold ΔP_thr for the active contraction pressure value ΔP.

The active contraction pressure value refers to a force generated per unit area when a muscle actively contracts.

In some embodiments, the EMG threshold Vₜₕᵣ and the contraction pressure threshold ΔP_thr may be preset based on experience. For example, the EMG threshold Vₜₕᵣ may be 8 μA, and the contraction pressure threshold ΔP_thr may be 12.3 cm H₂O.

In some embodiments, the processor may obtain the maximum value V_max of the EMG value V (i.e., a highest point of the test curve corresponding to the type II pelvic floor muscle fibers) and the peak contraction pressure ΔP_max of the pressure value P in the test segment corresponding to the type II pelvic floor muscle fibers. The peak contraction pressure ΔP_max is a maximum pressure value in the test curve corresponding to the type II pelvic floor muscle fibers minus a minimum pressure value.

The grade condition for the type II muscle refers to a condition used for grading the muscle strength for the type II muscle.

In some embodiments, in response to the evaluation metric being the EMG value, the grade condition for the type II muscle includes satisfying V_max>V_thr; in response to the evaluation metric being the pressure value, the grade condition for the type II muscle includes satisfying ΔP_max>ΔP_thr; in response to the evaluation metric including both the EMG value and the pressure value, the grade condition for type II includes simultaneously satisfying V_max>V_thr and ΔP_max>ΔP_thr. Furthermore, for the test curve, a portion below the threshold value is mostly the work of the type I muscle fibers, which is an irrelevant term and should be excluded. Therefore, the grade condition for the type II muscle also needs to include that at least one point in the test curve corresponding to the type II pelvic floor muscle fibers exceeds the threshold value Bou_val.

In some embodiments, in response to the grade condition for the type II muscle being satisfied, the determination of the muscle strength grade for the type II muscle is performed; in response to the grade condition not being satisfied, the result of the muscle strength grade for the type II muscle as a grade 0 is output.

In some embodiments of the present disclosure, introducing dual gating with the EMG threshold and the contraction pressure threshold before the muscle strength grade for the type II muscle, and setting a condition of “at least one point exceeding the threshold value” in the test curve corresponding to the type II pelvic floor muscle fibers, may help, in some cases, to shield segments with low signal-to-noise ratio caused by probe displacement, weak contractions, or environmental interference, thereby reducing the probability of misjudging valid type II muscle contractions. When the grade condition for the type II muscle is not satisfied, directly outputting the result of the muscle strength grade for the type II muscle as the grade 0 may help, in some cases, to terminate subsequent area ratio calculations early, reducing unnecessary computational consumption and improving a response speed of an overall evaluation process.

In some embodiments, determining the fatigue degree for type II muscle may include: selecting a third calculation point and a fourth calculation point in the test curve corresponding to the type II pelvic floor muscle fibers; and calculating the fatigue degree of the test curve corresponding to the type II pelvic floor muscle fibers using the fatigue degree calculation equation based on the third calculation point and the fourth calculation point.

In some embodiments, a first peak point of the test curve corresponding to the type II pelvic floor muscle fibers is selected and recorded as the third calculation point N₃, and a last peak point is selected and recorded as the fourth calculation point N₄. A third time point t₃ corresponding to the third calculation point N₃ and a fourth time point t₄ corresponding to the fourth calculation point N₄ are obtained, respectively.

In some embodiments, in response to the evaluation metric including the EMG value V of the vagina, a fatigue degree B₂ of the test curve corresponding to type II pelvic floor muscle fibers may be calculated using the EMG fatigue degree equation based on the EMG value corresponding to the third calculation point N₃ and the third time point t₃, and the EMG value corresponding to the fourth calculation point N₄ and the fourth time point t₄.

For example, the EMG fatigue degree equation may be a following equation (6):

$${B}_{2} = - 100 \% * \{ [ \vert {V}_{3} - {V}_{4} \vert \div {V}_{3} ] \div \vert {t}_{4} - {t}_{3} \vert \} \qquad ( 6 )$$

where, V₃ denotes an EMG value corresponding to the third calculation point N₃, and V₄ denotes an EMG value corresponding to the fourth calculation point N₄.

In some embodiments, in response to the evaluation metric including the pressure value P of the vagina, a fatigue degree B₂ of the test curve corresponding to type II pelvic floor muscle fibers may be calculated using the pressure fatigue degree equation based on the pressure value corresponding to the third calculation point N₃ and the third time point t₃, and the pressure value corresponding to the fourth calculation point N₄ and the fourth time point t₄.

For example, the pressure fatigue degree equation may be a following equation (7):

$${B}_{2} = - 100 \% * \{ [ \vert {P}_{3} - {P}_{4} \vert \div {P}_{3} ] \div \vert {t}_{4} - {t}_{3} \vert \} \qquad ( 7 )$$

where, P₃ denotes the pressure value corresponding to the third calculation point N₃, and P₄ denotes the pressure value corresponding to the fourth calculation point N₄.

Merely by way of example, if the test curve corresponding to the type II pelvic floor muscle fibers includes 5 peaks in total, a first peak point and a fifth peak point are taken as the third calculation point N₃ and the fourth calculation point N₄, respectively, and a slope is calculated to further characterize the fatigue degree for the type II muscle fibers.

In some embodiments of the present disclosure, the fatigue degree for the type II muscle fibers may be accurately determined by the above manner without manual subjective judgment, thereby reducing the subjective human influence and helping to improve the accuracy and efficiency of evaluating the test data of the pelvic floor muscle.

In some embodiments, the processor may also determine an optimal stimulation parameter based on the result of the muscle strength grade for the type I muscle and the result of the muscle strength grade for the type II muscle; and generate a stimulation instruction based on the optimal stimulation parameter.

The optimal stimulation parameter refers to a preferred parameter related to electrical stimulation applied to a user.

The stimulation instruction refers to an instruction determined based on the optimal stimulation parameter for instructing an electrical stimulation device.

In some embodiments, the optimal stimulation parameter include a stimulation duration and a rest interval. The stimulation instruction determined based on the optimal stimulation parameter also includes the stimulation duration and the rest interval.

The stimulation duration refers to a duration for which the electrical stimulation device stimulates the muscle each time.

The rest interval refers to an interval between each stimulation by the electrical stimulation device.

In some embodiments, the stimulation instruction is configured to control a neuromuscular electrical stimulation device to perform electrical stimulation on the user for a stimulation duration, and stop for a duration of the rest interval between each electrical stimulation.

The electrical stimulation device refers to an electronic device that stimulates nerves or muscles by applying a microcurrent to the skin surface to achieve different therapeutic or training purposes. In some embodiments, the electrical stimulation device may include a neuromuscular electrical stimulation device, a transcutaneous electrical nerve stimulation device, an electrical muscle stimulation device, a functional electrical stimulation device, a microcurrent device, etc.

In some embodiments, the processor may determine the optimal stimulation parameter by using a second vector database based on the result of the muscle strength grade for the type I muscle and the result of the muscle strength grade for the type II muscle. The processor may construct a second vector from the result of the muscle strength grade for the type I muscle and the result of the muscle strength grade for the type II muscle. The processor may obtain the optimal stimulation parameter by querying the second vector database based on the second vector.

The second vector database may include a plurality of reference second vectors and corresponding labels. The second vector database is constructed similarly to the first vector database. The reference second vector is constructed similarly to the second vector. The plurality of reference second vectors may be constructed from historical results of the muscle strength grade for the type I muscle and historical results of the muscle strength grade for the type II muscle corresponding to a plurality of historical stimulations. The label corresponding to the reference second vector is the optimal stimulation parameter corresponding to the reference second vector. The processor may obtain, from a plurality of historical users corresponding to the reference second vector, a historical stimulation parameter used by the historical user whose pelvic floor muscle function improved the most in a subsequent evaluation, and determine as the label corresponding to the reference second vector. The phrase “improved the most” regarding pelvic floor muscle function refers to a scenario where an average of increased levels of results of the muscle strength grade for the type I muscle and increased levels of results of the muscle strength grade for the type II muscle is the largest.

In some embodiments, the processor may select, from the second vector database, a reference second vector with the highest similarity to the second vector and a corresponding label of the reference second vector, as the optimal stimulation parameter corresponding to the second vector.

In some embodiments of the present disclosure, by automatically determining and controlling parameters of the electrical stimulation device based on quantified results of the muscle strength grade, a personalized and precise rehabilitation plan may be provided for the user.

In some embodiments of the present disclosure, the baseline images and test curves are constructed based on the test data of the pelvic floor muscle. Combining force generation features for the type I muscle fibers and the type II muscle fibers, and setting the threshold value, the muscle strength grade for the type I muscle is determined using a ratio of the contraction area of the test curve corresponding to the type I pelvic floor muscle fibers fibers to an area of a portion of the first baseline image not exceeding the threshold value. The muscle strength grade for the type II muscle is determined using a ratio of an area of a portion of the test curve corresponding to the type II pelvic floor muscle fibers fibers exceeding the threshold value to an area of a portion of the second baseline image exceeding the threshold value. Therefore, it enables automatic calculation of muscle strength grade for the type I muscle fibers and the type II muscle fibers. The fatigue degree determination may also be performed based on the baseline images and the test curves. Manual observation and judgment are not required. It allows for accurate and rapid determination of the muscle strength grade and determination of the fatigue degree for data of the pelvic floor muscle, reduces the subjective human influence, and helps improve the accuracy and efficiency for evaluating the test data of the pelvic floor muscle.

It should be noted that the foregoing descriptions of the process 100 is intended to be exemplary and illustrative only and do not limit the scope of application of the present disclosure. For those skilled in the art, various corrections and changes may be made to process 100 under the guidance of the present disclosure. However, these corrections and changes remain within the scope of the present disclosure.

One or more embodiments of the present disclosure also provide a system for evaluating the test data of the pelvic floor muscle. In some embodiments, the system for evaluating the test data of the pelvic floor muscle may include: an input module, a curve construction module, and a calculation module.

The input module is configured to obtain the test data of the pelvic floor muscle within a test period, wherein the test data includes an evaluation metric with temporal information, the evaluation metric includes an electromyography (EMG) value V and/or pressure value P of vagina, and the test data includes a test segment corresponding to type I pelvic floor muscle fibers and a test segment corresponding to type II pelvic floor muscle fibers.

The curve construction module is configured to preset a threshold value Bou_val, obtain a first baseline image for type I pelvic floor muscle fibers and a second baseline image for type II pelvic floor muscle fibers, construct a test curve corresponding to type I pelvic floor muscle fibers and a test curve corresponding to type II pelvic floor muscle fibers, respectively, based on the test segment corresponding to the type I pelvic floor muscle fibers and the test segment corresponding to the type II pelvic floor muscle fibers; wherein a planar Cartesian coordinate system is constructed with a test time as an X-axis and a percentage obtained by dividing an evaluation metric in the test segment corresponding to the type I pelvic floor muscle fibers by a maximum value of an evaluation metric in the first baseline image as a Y-axis, and a line formed in the system is the test curve corresponding to the type I pelvic floor muscle fibers; a planar Cartesian coordinate system is constructed with the test time as an X-axis and a percentage obtained by dividing an evaluation metric in the test segment corresponding to the type II pelvic floor muscle fibers by a maximum value of an evaluation metric in the second baseline image as a Y-axis, and a line formed in the system is the test curve corresponding to the type II pelvic floor muscle fibers.

The calculation module is configured to determine a muscle strength grade and/or a fatigue degree for the type I muscle by combining the first baseline image and the test curve corresponding to the type I pelvic floor muscle fibers; and determine a muscle strength grade and/or a fatigue degree for the type II muscle by combining the second baseline image and the test curve corresponding to the type II pelvic floor muscle fibers.

In some embodiments, the muscle strength grade for the type I muscle includes: designating a contraction area of the test curve corresponding to the type I pelvic floor muscle fibers as a first test area S_{A1}, designating an area of the first baseline image that does not exceed the threshold value Bouᵥₐₗ as a first reference area S_{B1}, and obtaining a result of the muscle strength grade for the type I muscle based on a first ratio F₁ of the first test area S_{A1} to the first reference area S_{B1}.

In some embodiments, the muscle strength grade for the type II muscle includes: designating a contraction area of the test curve corresponding to the type II pelvic floor muscle fibers as a first test area S_{A2}, designating an area of the second baseline image that does not exceed the threshold value Bouᵥₐₗ as a second reference area S_{B2}, and obtaining a result of the muscle strength grade for the type II muscle based on a second ratio F₂ of the second test area S_{A2} to the second reference area S_{B2}.

The system for evaluating pelvic floor muscle test data in the embodiments of the present disclosure shares the same inventive concept as the method for evaluating pelvic floor muscle test data described above. It can be understood with reference to the above description, and details are not repeated here.

The system for evaluating the test data of the pelvic floor muscle in the embodiments of the present disclosure may accurately and rapidly determine the muscle strength grade and fatigue degree calculation for pelvic floor muscle data, reduce subjective human influence, and improve the accuracy and efficiency for evaluating the test data of the pelvic floor muscle.

It should be understood that the system for evaluating the test data of the pelvic floor muscle and modules thereof may be implemented in various ways. For example, in some embodiments, the system and the modules thereof may be implemented by a processor.

It should be noted that the above description of the system for evaluating the test data of the pelvic floor muscle and the modules thereof is for descriptive convenience only and does not limit the present disclosure to the exemplified embodiments. It may be understood that for those skilled in the art, after understanding the principles of the system, various modules may be combined arbitrarily or constitute subsystems connected to other modules without departing from these principles. In some embodiments, the input module, the curve construction module, and the calculation module may be different modules in one system, or one module may implement the functions of two or more of the above modules. For example, the modules may share a storage module, or each module may have its own storage module. Such modifications fall within the protection scope of the present disclosure.

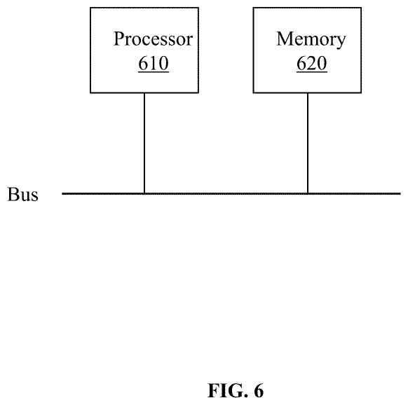

FIG. 6 is a schematic structural diagram illustrating a computer apparatus according to some embodiments of the present disclosure.

As shown in FIG. 6, in some embodiments, a computer apparatus includes a processor 610 and a memory 620 communicatively coupled. The memory 620 stores at least one instruction or at least one program. The at least one instruction or the at least one program is executed by the processor 610 when loaded to perform the method for evaluating the test data of the pelvic floor muscle as described above. The memory 620 may be used to store software programs and modules. The processor 610 executes various functional applications by running the software programs and modules stored in the memory 620. The memory 620 may mainly include a program storage area and a data storage area. The program storage area may store an operating system, application programs required for functions, etc. The data storage area may store data created according to the use of the apparatus, etc. In addition, the memory 620 may include a high-speed random access memory, and may also include a non-volatile memory, such as at least one magnetic disk storage device, a flash memory device, or other volatile solid-state storage devices. The memory 620 may further include a memory controller to provide the processor 610 with access to the memory 620.

The method embodiments provided by some embodiments of the present disclosure may be executed in a computer terminal, a server, or a similar computing device. That is, the computer apparatus may include a computer terminal, a server, or a similar computing device. The internal structure of the computer apparatus may include, but is not limited to, a processor, a network interface, and a memory. The processor, the network interface, and the memory in the computer apparatus may be connected via a bus or other means.

The processor 610 (also referred to as a Central Processing Unit, CPU) is the computing core and control core of the computer apparatus. The network interface may include a standard wired interface, a wireless interface (e.g., WI-FI, mobile communication interface, etc.). The memory 620 is a memory device in the computer apparatus, configured to store programs and data. The memory 620 may be a high-speed random access memory (RAM) storage device, or a non-volatile memory (NVM) storage device, such as at least one magnetic disk storage device. The memory 620 may also be at least one storage device located remotely from the processor 610. The memory 620 provides a storage space. The storage space stores an operating system of the electronic device, which may include, but is not limited to, a Windows system, a Linux system (GNU), an Android system, an IOS system, etc. In some embodiments, one or more instructions suitable to be loaded and executed by the processor 610 are also stored in the storage space. The instructions may be one or more computer programs and program codes. In some embodiments, the processor 610 loads and executes one or more instructions stored in the memory 620 to implement the method for evaluating the test data of the pelvic floor muscle described in the above method embodiments.

One or more embodiments of the present disclosure further provide a computer-readable storage medium. At least one instruction or at least one program is stored on the computer-readable storage medium. The at least one instruction or the at least one program, when loaded by the processor 610, causes the processor 610 to perform the method for evaluating pelvic floor muscle test data as described above. The computer-readable storage medium carries one or more programs. The method according to the embodiments of the present disclosure is implemented when the one or more programs are executed.

According to some embodiments of the present disclosure, the computer-readable storage medium may be a non-volatile computer-readable storage medium. Merely by way of example, the computer-readable storage medium includes, but is not limited to, a portable computer disk, a hard disk, a random access memory (RAM), a read-only memory (ROM), an erasable programmable read-only memory (EPROM or flash memory), a portable compact disk read-only memory (CD-ROM), an optical storage device, a magnetic storage device, etc., or any suitable combination thereof. In some embodiments, the computer-readable storage medium may be any tangible medium that contains or stores a program. The program may be used by or in conjunction with an instruction execution system, apparatus, or device.

Various other changes and modifications may be made by those skilled in the art based on the described technical solutions and concepts. All such changes and modifications shall fall within the scope of protection.

The method, system, and apparatus for evaluating the test data of the pelvic floor muscle provided by one or more embodiments of the present disclosure are based on the test data of the pelvic floor muscle to construct baseline images and test curves. Combining force generation features for the type I muscle fibers and the type II muscle fibers, and setting the threshold value, the muscle strength grade for the type I muscle is determined using a ratio of the contraction area of the test curve corresponding to the type I pelvic floor muscle fibers fibers to an area of a portion of the first baseline image not exceeding the threshold value. The muscle strength grade for the type II muscle is determined using a ratio of an area of a portion of the test curve corresponding to the type II pelvic floor muscle fibers fibers exceeding the threshold value to an area of a portion of the second baseline image exceeding the threshold value. Therefore, it enables automatic calculation of muscle strength grade for the type I muscle fibers and the type II muscle fibers. The fatigue degree determination may also be performed based on the baseline images and the test curves. Manual observation and judgment are not required. It allows for accurate and rapid determination of the muscle strength grade and determination of the fatigue degree for data of the pelvic floor muscle, reduces the subjective human influence, and helps improve the accuracy and efficiency for evaluating the test data of the pelvic floor muscle.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

1. A method for evaluating test data of pelvic floor muscle, comprising:
obtaining the test data of the pelvic floor muscle within a test period, wherein the test data includes an evaluation metric with temporal information, the evaluation metric includes an electromyography (EMG) value V and/or pressure value P of vagina, and the test data includes a test segment for type I muscle fibers and a test segment for type II muscle fibers;
presetting a threshold value Bou_val, obtaining a first baseline image for type I pelvic floor muscle fibers and a second baseline image for type II pelvic floor muscle fibers, constructing a test curve for type I muscle and a test curve for type II muscle, respectively, based on the test segment for the type I muscle fibers and the test segment for the type II muscle fibers; wherein a planar Cartesian coordinate system is constructed with a test time as an X-axis and a percentage obtained by dividing an evaluation metric in the test segment for the type I muscle fibers by a maximum value of an evaluation metric in the first baseline image as a Y-axis, and a line formed in the system is the test curve corresponding to the type I pelvic floor muscle fibers; a planar Cartesian coordinate system is constructed with the test time as an X-axis and a percentage obtained by dividing an evaluation metric in the test segment for the type II muscle fibers by a maximum value of an evaluation metric in the second baseline image as a Y-axis, and a line formed in the system is the test curve corresponding to the type II pelvic floor muscle fibers;
determining a muscle strength grade and/or a fatigue degree for the type I muscle by combining the first baseline image and the test curve corresponding to the type I pelvic floor muscle fibers; and determining a muscle strength grade and/or a fatigue degree for the type II muscle by combining the second baseline image and the test curve corresponding to the type II pelvic floor muscle fibers;
wherein the muscle strength grade for the type I muscle includes: designating a contraction area of the test curve corresponding to the type I pelvic floor muscle fibers as a first test area S_{A1}, designating an area of the first baseline image that does not exceed the threshold value Bou_val as a first reference area S_{B1}, and obtaining a result of the muscle strength grade of the test curve corresponding to the type I pelvic floor muscle fibers based on a first ratio F₁ of the first test area S_{A1} to the first reference area S_{B1};
the muscle strength grade for the type II muscle includes: designating a contraction area of the test curve corresponding to the type II pelvic floor muscle fibers as a first test area S_{A2}, designating an area of the second baseline image that does not exceed the threshold value Bou_val as a second reference area S_{B2}, and obtaining a result of the muscle strength grade of the test curve corresponding to the type II pelvic floor muscle fibers based on a second ratio F₂ of the second test area S_{A2} to the second reference area S_{B2};
discretizing the test curve corresponding to the type I pelvic floor muscle fibers into n₁ consecutive data points in the muscle strength grade for the type I muscle, converting the first baseline image as a trapezoid-approximated image, and determining the first ratio F₁ of the first test area S_{A1} to the first reference area S_{B1} by following equation (1):
$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \% \qquad ( 1 )$$
wherein k denotes an ordinal number of a data point, k∈[1, n₁], pointₖ denotes a Y-axis corresponding to a k-th data point, t denotes a test duration of the test curve corresponding to the type I pelvic floor muscle fibers, D₁ and D₂ denote an upper base and lower base of a trapezoidal image, respectively, h₁ denotes a height of the trapezoidal image, and h₁ is equal to the threshold value Bou_val;
in the muscle strength grade for the type I muscle:
in response to the first ratio F₁∈[0%, 10%], outputting the result of the muscle strength grade for the type I muscle as a grade 0;
in response to the first ratio F₁∈(10%, 28%], outputting the result of the muscle strength grade for the type I muscle as a grade 1;
in response to the first ratio F₁∈(28%, 46%], outputting the result of the muscle strength grade for the type I muscle as a grade 2;
in response to the first ratio F₁∈(46%, 64%], outputting the result of the muscle strength grade for the type I muscle as a grade 3;
in response to the first ratio F₁∈(64%, 82%], outputting the result of the muscle strength grade for the type I muscle as a grade 4; and
in response to the first ratio F₁∈(82%, 100%], outputting the result of the muscle strength grade for the type I muscle as a grade 5.

2. The method of claim 1, wherein before the muscle strength grade for the type II muscle, the method further includes:
presetting an EMG threshold Vₜₕᵣ of the EMG value V and a contraction pressure threshold ΔP_thr of an active contraction pressure value ΔP, obtaining a maximum value V_max of the EMG value and a peak contraction pressure ΔP_max of the pressure value P in the test segment for type II muscle fibers;
in response to a grade condition for the type II muscle being satisfied, performing the muscle strength grade for the type II muscle; wherein the grade condition for the type II muscle includes V_max>V_thr, ΔP_max>ΔP_thr, and at least one point in the test curve corresponding to the type II pelvic floor muscle fibers exceeds the threshold value Bou_val; and
in response to the grade condition for the type II muscle not being satisfied, outputting the result of the muscle strength grade for the type II muscle as a grade 0.

3. The method of claim 1, wherein the determining the fatigue degree for the type I muscle includes:
selecting a first peak point that exceeds a screening threshold in an initial segment of the test curve corresponding to the type I pelvic floor muscle fibers as a first calculation point N₁, selecting another point after a certain time period following the first calculation point N₁ as a second calculation point N₂, obtaining a first time point t₁ corresponding the first calculation point N₁ and a second time point t₂ corresponding the second calculation point N₂;
in response to the evaluation metric including the EMG value V of the vagina, determining a fatigue degree B₁ of the test curve corresponding to the type I pelvic floor muscle fibers as following equation (2):
$${B}_{1} = - 100 \% * \{ [ \vert {V}_{1} - {V}_{2} \vert \div {V}_{1} ] \div \vert {t}_{2} - {t}_{1} \vert \} \qquad ( 2 )$$
wherein V₁ denotes an EMG value corresponding to the first calculation point N₁, and V₂ denotes an EMG value corresponding to the second calculation point N₂;
in response to the evaluation metric including the pressure value P of the vagina, the fatigue degree B₁ of the test curve corresponding to the type I pelvic floor muscle fibers as following equation (3):
$${B}_{1} = - 100 \% * \{ [ \vert {P}_{1} - {P}_{2} \vert \div {P}_{1} ] \div \vert {t}_{2} - {t}_{1} \vert \} \qquad ( 3 )$$
wherein P₁ denotes a pressure value corresponding to the first calculation point N₁, and P₂ denotes a pressure value corresponding to the second calculation point N₂.

4. The method of claim 1, in the muscle strength grade for the type II muscle, wherein the method further includes:
dividing a test duration of the test curve corresponding to the type II pelvic floor muscle fibers into m consecutive data intervals evenly, wherein each data interval includes n₂ consecutive data points, obtaining a maximum baseline EMG value V_ref_max, a minimum baseline EMG value V_ref_min, a maximum baseline pressure value P_ref_max, and a minimum baseline pressure value P_ref_min, and determining a ratio F′ of the second test area S_{A2} to the second reference area S_{B2} by following equations (4) and (5):
$${F}^{′} = \frac{{S}_{A 2}}{{S}_{B 2}} = \frac{{\sum }_{k = i}^{i + {n}_{2}} {pro}_{k}}{\frac{\frac{l}{T} * {n}_{2} * {h}_{2}}{2}} * 100 \% \qquad ( 4 )$$
$${pro}_{k} = \frac{V\_k - V\_ref \_min}{V\_ref \_max - V\_ref \_min} * 100 \% or \frac{P\_k - P\_ref \_min}{P\_ref \_max - P\_ref \_min} * 1 \; 00 \% ; \qquad ( 5 )$$
wherein i denotes a number of a first data point in the data interval, k denotes the ordinal number of the data point, k∈[1, m*n₂], V_k denotes an EMG value corresponding to the k-th data point, P_k denotes a pressure value corresponding to the k-th data point, I denotes a base length of a portion of the second baseline image where values exceed the threshold value Bou_val, T denotes a time length of each data interval, and h₂ denotes a height of a portion of the second baseline image where values exceed the threshold value Bou_val;
summing the ratio F′ of the second test area S_{A2} to the second reference area S_{B2} of each data interval and calculating a mean value of the sum to obtain a second ratio F₂;
in the muscle strength grade for the type II muscle,
in response to the second ratio F₂∈(0%, 20%], outputting the result of the muscle strength grade for the type II muscle as a grade 1;
in response to the second ratio F₂∈(20%, 40%], outputting the result of the muscle strength grade for the type II muscle as a grade 2;
in response to the second ratio F₂∈(40%, 60%], outputting the result of the muscle strength grade for the type II muscle as a grade 3;
in response to the second ratio F₂∈(60%, 80%], outputting the result of the muscle strength grade for the type II muscle as a grade 4; and
in response to the second ratio F₂ € (80%, 100%], outputting the result of the muscle strength grade for the type II muscle as a grade 5.

5. The method of claim 1, wherein the determining the fatigue degree for the type II muscle includes:
selecting a first peak point of the test curve corresponding to the type II pelvic floor muscle fibers as a third calculation point N₃, selecting a last point of the test curve corresponding to the type II pelvic floor muscle fibers as a fourth calculation point N₄, obtaining a third time point t₃ corresponding the third calculation point N₃ and a fourth time point t₄ corresponding the fourth calculation point N₄;
in response to the evaluation metric including the EMG value V of the vagina, determining a fatigue degree B₂ of the test curve corresponding to the type II pelvic floor muscle fibers as following equation (6):
$${B}_{2} = - 100 \% * \{ [ \vert {V}_{3} - {V}_{4} \vert \div {V}_{3} ] \div \vert {t}_{4} - {t}_{3} \vert \} \qquad ( 6 )$$
wherein V₃ denotes an EMG value corresponding to the third calculation point N₃, and V₄ denotes an EMG value corresponding to the fourth calculation point N₄;
in response to the evaluation metric including the pressure value P of the vagina, the fatigue degree B₂ of the test curve corresponding to the type I pelvic floor muscle fibers as following equation (7):
$${B}_{2} = - 100 \% * \{ [ \vert {P}_{3} - {P}_{4} \vert \div {P}_{3} ] \div \vert {t}_{4} - {t}_{3} \vert \} \qquad ( 7 )$$
wherein P₃ denotes a pressure value corresponding to the third calculation point N₃, and P₄ denotes a pressure value corresponding to the fourth calculation point N₄.

6. The method of claim 1, wherein the threshold value Bouᵥₐₗ is 50%.

7. A system for evaluating test data of pelvic floor muscle, comprising:
an input module, configured to obtain the test data of the pelvic floor muscle within a test period, wherein the test data includes an evaluation metric with temporal information, the evaluation metric includes an electromyography (EMG) value V and/or pressure value P of vagina, and the test data includes a test segment for type I muscle fibers and a test segment for type II muscle fibers;
a curve construction, configured to preset a threshold value Bou_val, obtain a first baseline image for type I pelvic floor muscle fibers and a second baseline image for type II pelvic floor muscle fibers, construct a test curve for type I muscle and a test curve for type II muscle, respectively, based on the test segment for the type I muscle fibers and the test segment for the type II muscle fibers; wherein a planar Cartesian coordinate system is constructed with a test time as an X-axis and a percentage obtained by dividing an evaluation metric in the test segment for the type I muscle fibers by a maximum value of an evaluation metric in the first baseline image as a Y-axis, and a line formed in the system is the test curve corresponding to the type I pelvic floor muscle fibers; a planar Cartesian coordinate system is constructed with the test time as an X-axis and a percentage obtained by dividing an evaluation metric in the test segment for the type II muscle fibers by a maximum value of an evaluation metric in the second baseline image as a Y-axis, and a line formed in the system is the test curve corresponding to the type II pelvic floor muscle fibers;
a calculation module, configured to determine a muscle strength grade and/or a fatigue degree for the type I muscle by combining the first baseline image and the test curve corresponding to the type I pelvic floor muscle fibers; and determine a muscle strength grade and/or a fatigue degree for the type II muscle by combining the second baseline image and the test curve corresponding to the type II pelvic floor muscle fibers; wherein
the muscle strength grade for the type I muscle includes: designating a contraction area of the test curve corresponding to the type I pelvic floor muscle fibers as a first test area S_{A1}, designating an area of the first baseline image that does not exceed the threshold value Bouᵥₐₗ as a first reference area S_{B1}, and obtaining the muscle strength grade of the test curve corresponding to the type I pelvic floor muscle fibers based on a first ratio F₁ of the first test area S_{A1} to the first reference area S_{B1};
the muscle strength grade for the type II muscle includes: designating a contraction area of the test curve corresponding to the type II pelvic floor muscle fibers as a first test area S_{A2}, designating an area of the second baseline image that does not exceed the threshold value Bouᵥₐₗ as a second reference area S_{B2}, and obtaining the muscle strength grade of the test curve corresponding to the type II pelvic floor muscle fibers based on a second ratio F₂ of the second test area S_{A2} to the second reference area S_{B2};
discretizing the test curve corresponding to the type I pelvic floor muscle fibers into n₁ consecutive data points in the muscle strength grade for the type I muscle, converting the first baseline image as a trapezoid-approximated image, and determining the first ratio F₁ of the first test area S_{A1} to the first reference area S_{B1} by following equation (1):
$${F}_{1} = \frac{{S}_{A 1}}{{S}_{B 1}} = \frac{{\sum }_{k = 1}^{{n}_{1}} {point}_{k}}{\frac{1}{2} * ( \frac{1}{t} * ( {D}_{1} + {D}_{2} ) * n * {h}_{1} )} * 100 \% \qquad ( 1 )$$
wherein k denotes an ordinal number of a data point, k∈[1, n₁], pointₖ denotes a Y-axis corresponding to a k-th data point, t denotes a test duration of the test curve corresponding to the type I pelvic floor muscle fibers, D₁ and D₂ denote an upper base and lower base of a trapezoidal image, respectively, h₁ denotes a height of the trapezoidal image, and h₁ is equal to the threshold value Bou_val;
in the muscle strength grade for the type I muscle:
in response to the first ratio F₁∈[0%, 10%], outputting a result of the muscle strength grade for the type I muscle as a grade 0;
in response to the first ratio F₁∈(10%, 28%], outputting a result of the muscle strength grade for the type I muscle as a grade 1;
in response to the first ratio F₁∈(28%, 46%], outputting a result of the muscle strength grade for the type I muscle as a grade 2;
in response to the first ratio F₁∈(46%, 64%], outputting a result of the muscle strength grade for the type I muscle as a grade 3;
in response to the first ratio F₁∈(64%, 82%], outputting a result of the muscle strength grade for the type I muscle as a grade 4; and
in response to the first ratio F₁∈(82%, 100%], outputting a result of the muscle strength grade for the type I muscle as a grade 5.

8. A computer apparatus, comprising a processor and a memory in signal communication, wherein the memory stores at least one instruction or at least one program segment, and the processor, when loading the at least one instruction or the at least one program segment, is configured to execute the method for evaluating test data of pelvic floor muscle according to claim 1.

9. A computer-readable storage medium, storing at least one instruction or at least one program segment, wherein the at least one instruction or the at least one program segment, when loaded and executed by a processor, causes the processor to perform the method for evaluating test data of pelvic floor muscle according to claim 1.
